(19) United States
(12) Patent Application Publication
(10) Pub. No.: US 2024/0277414 A1
(43) Pub. Date: Aug. 22, 2024

(54) BONY LANDMARK DETERMINATION SYSTEMS AND METHODS

(71) Applicant: DePuy Ireland Unlimited Company, Ringaskiddy (IE)

(72) Inventors: Daniel Girardeau-Montaut, Gieres (FR); Nicolas Demanget, Saint-Egreve (FR)

(21) Appl. No.: 18/112,863

(22) Filed: Feb. 22, 2023

(51) Int. Cl. A61B 34/20; A61B 34/30; G06T 7/60; G06T 7/73; G06T 11/00

(52) U.S. Cl. CPC A61B 34/20; A61B 34/30; G06T 7/60; G06T 7/74; G06T 11/00; A61B 2034/2065; A61B 2090/363; G06T 2207/10028; G06T 2207/30008; G06T 2210/41; G06T 2210/56

(57) ABSTRACT

Systems and methods are disclosed comprising receiving positional information comprising a plurality of acquired data points associated with a surface of an anatomical feature of a patient, determining a point cloud based on the plurality of data points, extracting a point from the plurality of data points as indicating a landmark on the anatomical feature, determining a boundary associated with the plurality of data points, comparing a distance between the boundary and the landmark point to a predetermined threshold, wherein if the distance between the boundary and the landmark point is not within the predetermined threshold, generating an indication requiring confirmation of the landmark point or a suggestion to acquire additional data points.

### BACKGROUND

Detection of certain anatomical features of a patient (e.g., such as on a patient's bone) is beneficial in planning a number of surgical procedures. For example, in a robotic or robot-assisted surgical procedure, in which a surgical robot may guide or control one or more surgical instruments (such as a saw, drill, etc.), precise determination of the location and/or orientation of the bone is desirable. This is particularly true in so-called imageless techniques, where no imaging-derived 3D model of the patient's bone is created.

Instead of relying on preoperative images, in an imageless system, a pointer and tracking system are used intraoperatively to acquire predetermined anatomical landmarks. Examples of such landmarks are anatomical lines or certain anatomical features. In the case of bone, the acquired bony landmarks are then used in navigation to create an initial intraoperative planning proposal that the surgeon can adjust if needed. Using implant planning data, cutting planes or tool trajectories for the robotic or robot-assisted surgical procedure are computed and this information is used to help the surgeon to navigate instruments in order to better position the implant and hopefully improve patient's outcomes.

In applications such as knee surgery (e.g., total knee arthroplasty (TKA) or uni-compartmental knee arthroplasty (UKA)) or hip surgery (e.g., hip arthroplasty), the bony landmarks to be acquired are well known. However, actual acquisition may be problematic. For example, a system may require a surgeon to indicate when a tip of a pointer or probe is in proximity to the bony landmark. Error may be introduced in several ways, as will be described, for example, accessibility of the bone may be limited.

Accordingly, there is a need for improved systems and methods to be used during a surgical procedure.

### SUMMARY

Systems and methods are disclosed comprising receiving positional information comprising a plurality of acquired data points associated with a surface of an anatomical feature of a patient, determining a point cloud based on the plurality of data points, extracting a point from the plurality of data points as indicating a landmark on the anatomical feature, determining a boundary associated with the plurality of data points, comparing a distance between the boundary and the landmark point to a predetermined threshold, wherein if the distance between the boundary and the landmark point is not within the predetermined threshold, generating an indication requiring confirmation of the landmark point or a suggestion to acquire additional data points.

### BRIEF DESCRIPTION OF THE DRAWINGS

FIG. 1A is a schematic of a robotic surgical system;

FIG. 1B is a schematic of another robotic surgical system with a movable base cart;

FIG. 2 is a schematic of a system comprising a controller configured to select a robust landmark point;

FIG. 3A is a schematic of a bone and a plurality of acquired points;

FIG. 3B is a schematic of FIG. 3A with an extracted landmark point;

FIG. 4A is another schematic of a bone and a plurality of acquired points with an extracted landmark point;

FIG. 4B is a schematic of FIG. 4A from a different perspective; and

FIG. 4C is a schematic of FIG. 4A with a new extracted landmark point.

### DETAILED DESCRIPTION

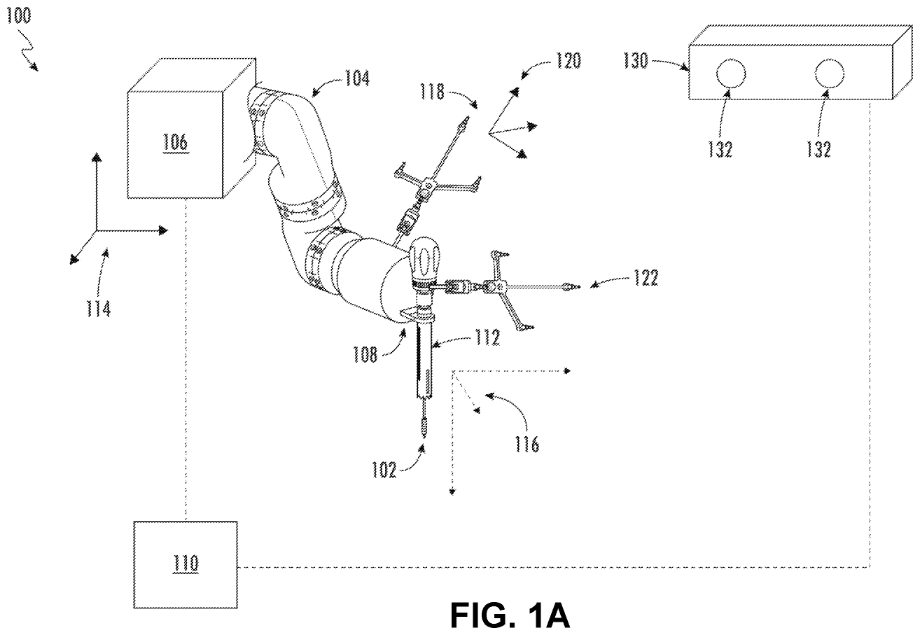

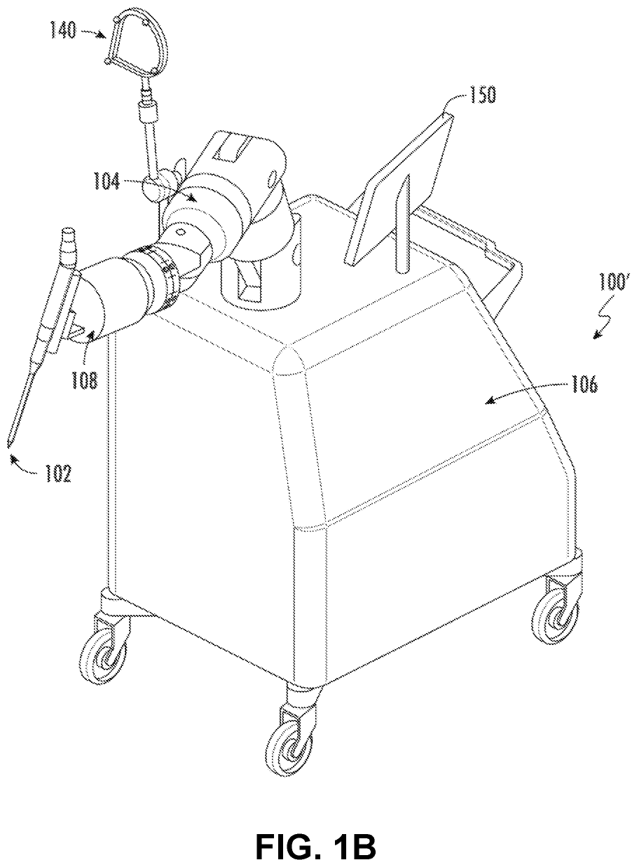

FIG. 1A illustrates a robotic (e.g., robot-assisted) surgical system 100 that may be utilized with the systems and methods described herein or after the bony landmark(s) have been acquired. An instrument 102 (which may also be referred to as a surgical tool) may be controlled by the system. Examples of instruments include, for example, a drill bit, saw blade, burr, reamer, mill, knife, or any other implement that could cut or deform bone or other tissue (e.g., penetrate) and is appropriate for use in a given operation (e.g., a drill may be more appropriate in one operation while a saw may be more appropriate in another operation, etc.). The system 100 comprises a robotic arm 104 extending from a base 106, and terminating in an end effector 108 for attaching to the instrument 102. The robotic arm 104 has a plurality of arm segments connected by rotatable joints, the movement of which may be controller by a control system, referred to herein as a controller 110.

The controller 110 may be used to actuate the robotic arm 104 (e.g., control the actuation of each joint) to control movement and thus position the end effector 108, which effects a trajectory of the instrument 102. The controller 110 typically includes power supply, AC/DC converters, motion controllers to power the motors of the actuation units in each joint, fuses, real-time control system interface circuits, and other components included in surgical robot devices. The controller 110 is also configured to perform the systems and methods described herein regarding acquisition of the bony landmark(s). The controller 110 may also be configured to send an alert (such as of an indication requiring confirmation of the bony landmark point or a suggestion to acquire additional data points). The controller 110 may also be configured to make determinations as will be described.

The end effector 108 may comprise an instrument mount or guide 112 configured to receive the instrument 102. Further, the present disclosure is also contemplated to include use of such instruments by surgical robots, by users with some degree of robotic assistance, and in some cases perhaps without involvement of surgical robots or robotic assistance (e.g., but with a controller configured to determine, based on the data, at least one bony landmark point).

While the illustrated embodiments and accompanying description may refer to a specific surgery, the systems and methods described herein may be utilized in various applications involving robotic, robot-assisted, and non-robotic operations where computer-assisted instrument location are desired and precise adjustment of instrument position may be appropriate. Example applications include knee surgery (e.g., total knee arthroplasty (TKA) or uni-compartmental knee arthroplasty (UKA)), hip surgery (e.g., hip arthroplasty), shoulder surgery, spine surgery, and other orthopedic surgeries. The teachings of the present disclosure may be applied to such procedures; however, the systems and methods described herein are not limited to these applications.

The robot-assisted surgical system 100 may have a plurality of navigational features to determine a position and orientation in absolute space (with respect to all degrees of freedom of a three-dimension coordinate system), thereby determining a trajectory of the instrument 102. For example, the robotic device as a whole may be said to have a global coordinate system 114, which may be defined in different ways, but generally uses the location of the base 106. A position of various components may be determined, for example, calculated by receiving a position signal from an encoder in each joint of the robotic arm 104. For example, the end effector 108 may be constrained to move about such that the summation of the positions of the joints defines the location of an end effector coordinate system 116 in the global coordinate system 114.

Position may (e.g., may also) be directly measured. A navigation array 118 may be mounted to a distal portion of the robotic arm 104. The navigation array 118 may include one or more markers or sensors in a unique constellation or geometric arrangement. For example, optical navigation or tracking systems may utilize stereoscopic sensors to detect light emitting diodes (LEDs) or infra-red (IR) light reflected or emitted from one or more optical markers affixed to the array. For example, when the markers are reflective elements, once detected by stereoscopic sensors, the relative arrangement of the elements in the sensors' field of view, in combination with the known geometric arrangement of the elements, may allow the system to determine a three-dimensional position and orientation of the array. Other examples of tracking systems in include ultrasonic sensors, radio-frequency identification (RFID) sensors or other radio frequency (RF) tracking systems, electromagnetic interference (EMI) tracking systems, etc.

In some instances, a measured coordinate system 120 of the array 118 may be used as the global coordinate system 114. A navigation array 122 (additionally or alternatively to the array 118) may be mounted to the instrument 102 or the end effector 108. The navigation array 122 may include one or more markers, for example, in a unique constellation or geometric arrangement. The controller 110 may (e.g., in conjunction with the navigation system) use the markers to determine a three-dimensional position of the end effector 108 and/or the instrument 102.

The end effector coordinate system 116 may be defined in different ways, but may refer to the position and orientation of the end effector 108 with respect to the operation of the instrument 102. The array 122 may identify a positioning of the instrument 102. In this manner, the array 122 may help provide complete positioning information (e.g., of the instrument 102), which may be used by the controller (e.g., a surgical robot system, surgeons, etc.).

A navigation (e.g., tracking) system comprising the arrays and a tracking unit 130 may be provided so that the relative pose or three-dimensional position and orientation of the array 118 and/or 122, as well as any other navigation arrays present in an operating theater, e.g., such as an array coupled to patient anatomy (not shown), a surgical table (not shown), or a pointer (as will be described), may be tracked and shared to the controller 110 and any additional planning system. In some embodiments, the tracking unit 130 may include one or more navigation system cameras 132 that may capture a location of the one or more markers in the arrays 118 and/or 122.

The tracking unit 130 may measure the relative motions between any and all coordinate systems in real time. Real time can, in some embodiments, mean high frequencies greater than twenty Hertz, in some embodiments in the range of one hundred to five hundred Hertz, with low latency, in some embodiments less than five milliseconds. The location information captured from the markers of an array may thus identify a location of the component to which it is coupled in three-dimensional space given the known and precise relationship between the array and the component. For example, the array 122 may be configured to identify the 3D position of the instrument 102, such as a tip, without being permanently connected or fastened to the instrument.

In a manner similar to the arrays discussed above, a further array (not shown) may be coupled with a patient or other structure in the operating environment (e.g., a surgical table, etc.), to assist with keeping tracking of an anatomy of interest, for example, a femur, tibia, or a pedicle of the spine. A patient coordinate system may be defined in different ways (e.g., using an array coupled to the patient), but may refer to the position and orientation of the patient with respect to the end effector 108 or instrument 102. The navigation system (e.g., such as the tracking unit 130) may track these objects for purposes of displaying their relative positions and orientations to the surgeon and, in some cases, for purposes of controlling and/or constraining manual manipulation of the instrument relative to virtual boundaries associated with the patient's anatomy.

FIG. 1B illustrates another embodiment of a system 100′ that may be used with a surgical robot device 100′. The surgical system of FIG. 1B may be similar to the surgical system of FIG. 1A, in that it may include a robotic arm 104 having multiple arm segments joined together by a plurality of joints and a sensor-equipped instrument 102. The robotic arm 104 may be coupled to a movable cart at its base 106. Additionally, one or more navigation arrays 140 may be coupled to various parts of the robot device 100. Only representative array 140 is shown, but a plurality of arrays and a navigation system may be employed as discussed above in connection with FIG. 1A. An external device 150 may communicate with the controller (not depicted). The device 150 may be a display, a computing device, remote server, etc., configured to allow a surgeon or other user to input data directly into the controller. Such data may include patient information and/or surgical procedure information. The device 150 may display information from the controller, such as alerts or notification that instrument use has been ceased (e.g., via a closed loop command from the controller). Communication between the device 150 and the controller may be wireless (e.g., near-field communication (NFC), Wi-Fi, Bluetooth, Bluetooth LE, ZigBee, and the like) or wired (e.g., USB or Ethernet).

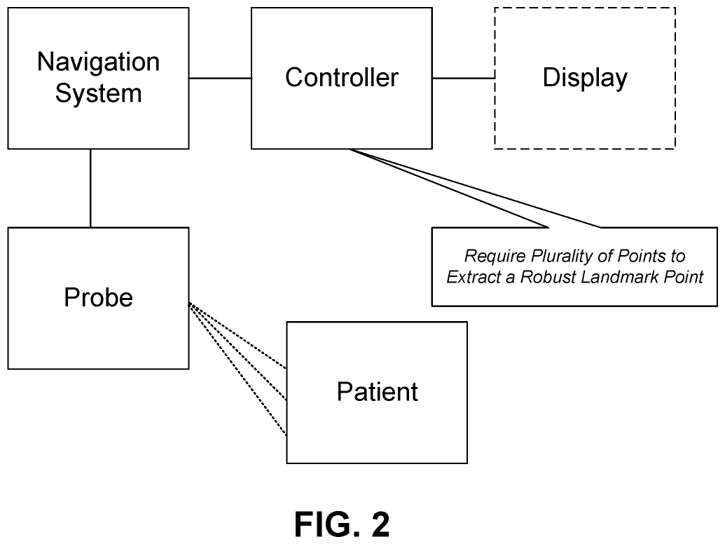

Turning to FIG. 2, in an imageless system, a pointer and navigation (e.g., tracking) system may be used intraoperatively to acquire positional information about predetermined anatomical landmark(s). The controller (such as controller 110 of FIG. 1A, although the disclosure contemplates embodiments without any robotic assistance) may communicate with the navigation system and an optional display to prompt a user (e.g., a surgeon) to position a pointer (e.g., a probe) at various anatomical landmarks on a patient relevant to a surgical procedure. The controller may be configured to derive a robust estimation of the location of the anatomical landmark(s), and extract a landmark point, as will be described.

The pointer may be equipped with a tracker (such as the array 122 (FIG. 1A)). The tracker may be mounted on the pointer (e.g., integrally or removably). The navigation system and/or the controller may utilize a known fixed geometric relationship between elements of the tracker to determine a precise three-dimensional position and orientation of the pointer. Examples of tracking systems include optical tracking systems with reflective markers, radio frequency (RF) tracking systems, electromagnetic interference (EMI) tracking systems, etc. A distance and orientation between the pointer tip and the tracker may be predetermined, so that a 3D position of the tip may be determined and stored as a point. In a preferred embodiment, the tip of the pointer is placed in contact with the bone. The surgeon may actively indicate that the pointer is in place, such as by voice command, depressing a foot pedal, pressing a button on the pointer, etc. Alternatively, the surgeon may passively indicate that the pointer is in place, such as by holding a position past expiry of a timer in the controller. Alternatively, the surgeon may move a distal portion of the pointer body in a predetermined pattern (such as a circle) to trigger acquisition of a point (e.g., while maintaining the point tip in place).

Optionally, a second tracker having a fixed geometric relationship may be coupled to a portion of patient anatomy or a surface in the surgical theater. The second tracker may employ the same types of markers as the pointer. The second tracker may represent a global coordinate system, for example, with reference to the patient. In some embodiments, the pointer tracker is dynamic (e.g., moving and detected at regular intervals) and the patient tracker is rigidly attached to the patient's bone.

Bony landmarks (e.g., anatomical positions on the patient that a surgeon or other user will be prompted to acquire) may be predetermined and stored in the controller. As may be appreciated, different landmarks are used for different surgeries. The controller may prompt the surgeon or other user to select a type of surgery before displaying a landmark to be acquired.

Example applications include knee surgery, e.g., total knee arthroplasty (TKA) or uni-compartmental knee arthroplasty (UKA), hip surgery, e.g., hip arthroplasty, shoulder surgery, spine surgery, etc. For example, in a hip arthroplasty, the landmark may relate to the anterior superior iliac spine, the posterior superior iliac spine, or a series of points to establish an Anterior Pelvic (AP) planar coordinate system. In another example, the landmark may relate to a set of individual points (e.g., the medial tibia malleolus, the lateral tibia malleolus, etc.) or a line (e.g. the anterior aspect of the femur) or a portion of surface (e.g. some specific parts or even the whole femur or tibia cartilage articular surface). In yet another example, in knee arthroplasty, the landmark may relate to some portions of the femur condyles surfaces (e.g., the distal and posterior portions of both the medial and lateral condyles).

The controller may be configured to require a plurality of acquired points before determining a landmark point. For example, the controller may take the plurality of points and create a 3D point cloud. In the case of a knee arthroplasty procedure, the controller may be configured to extract a single point (e.g., the landmark point) from each point cloud (e.g., the most distal point of the 3D point cloud for the distal portion of the condyles, and the most posterior point for the posterior portion of the condyles). Accuracy of an imageless procedure may be improved, such as compared to a system where a surgeon or other user is asked to manually pick a single landmark point. In some preferred embodiments, the landmark point is one of the points actually acquired by the surgeon (the controller is configured to extract one of the acquired points and deem it the landmark point). The controller may implement additional queries to the surgeon (e.g., verification and validation).

The controller may determine the landmark point without the use of preoperative images, such as computed tomography (CT), magnetic resonance imaging (MRI), ultrasound, other three-dimensional (3D) images, 2D images merged to afford a 3D image (e.g., fluoroscopy), or 2D images such as X-rays.

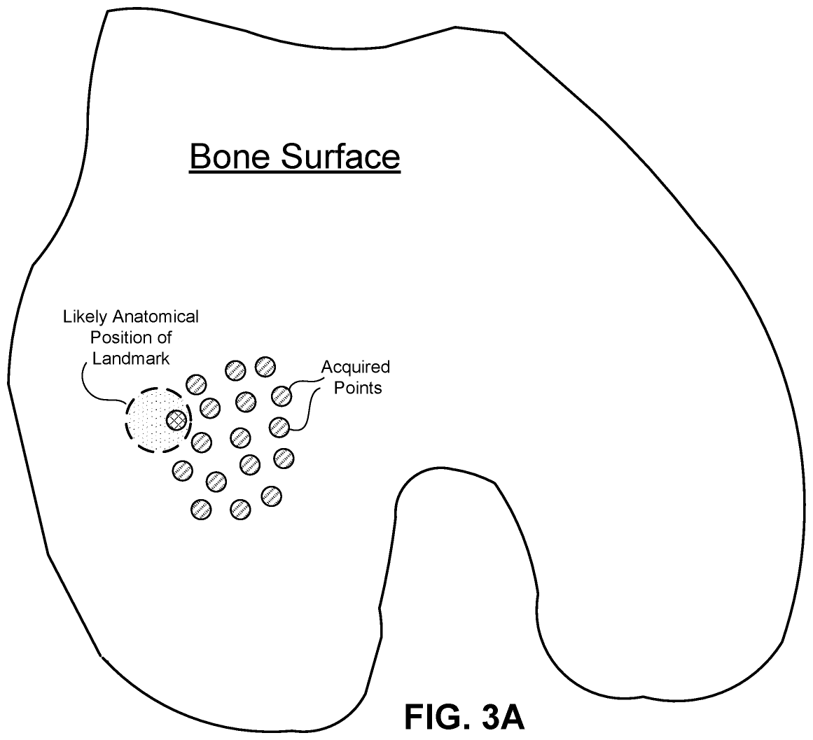

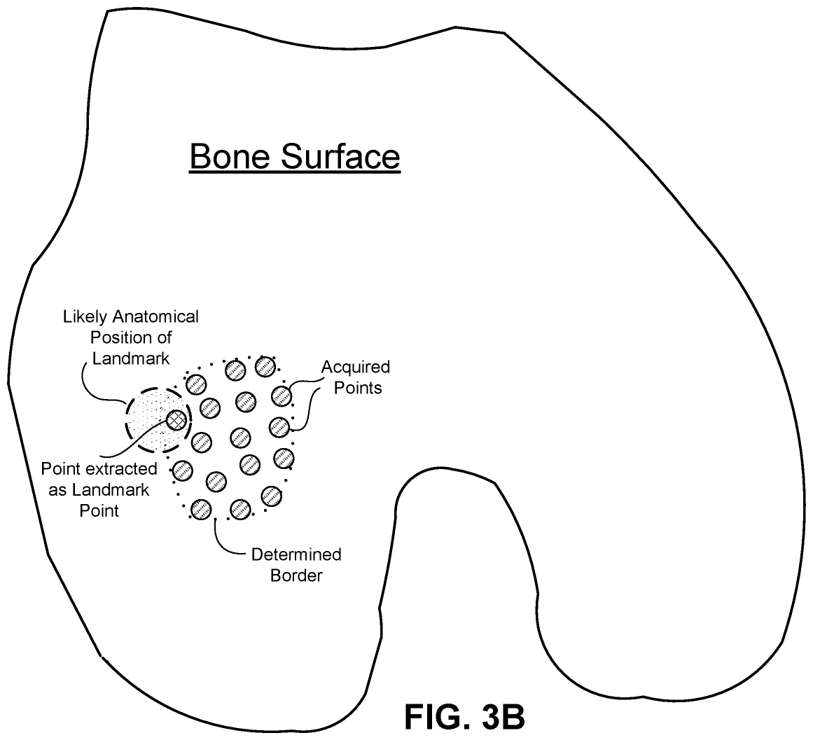

FIG. 3A is a schematic of a bone and a plurality of acquired points. As may be appreciated, every patient's bone is somewhat unique, however, given similarity in anatomy, there is a likely anatomical position of a landmark (e.g., an anatomical place). A controller, such as described herein, may receive input regarding a type of surgical procedure. The controller may determine to request a plurality of acquired points associated with a surface of an anatomical feature of a patient. For example, in a knee arthroplasty procedure, such as a UKA, a surgeon may need to acquire a most distal position and/or most posterior position of an anatomical feature as a landmark. The system may be used intraoperatively to acquire positions of predetermined anatomical landmarks.

A tracked pointer may be placed in contact with a surface of the bone by the surgeon at an anatomical landmark. The surgeon may acquire a plurality of points as described previously, for example, actively or passively indicating that the pointer is in contact with the bone. For each place where the pointer is indicated as being in contact with the bone, the controller may determine positional information for each place and store the positional information as an acquired point. Preferably, a plurality of acquired points are stored. It may be appreciated that despite having a plurality of acquired points, the portion of surface acquired by the surgeon might not be wide enough or properly positioned so as to encompass the actual most distal feature or most posterior feature of the anatomy. As seen in FIG. 3, the acquired points are not centered around the likely anatomical position of the landmark. This may be a result of insufficient coverage of the likely anatomical position of the landmark (e.g., coverage of the condyle surface) or it may be sufficient. Advantageously, the controller may be configured not only to extract a landmark point, but to check its robustness and trigger a potential warning. Alternatively, points may be acquired using a contact-less method (for example, a laser scanner or a white light scanner).

Turning to FIG. 3B, the controller is configured to create a point cloud with the acquired points. The point cloud may represent an acquired surface patch. The controller may determine a boundary associated with the plurality of acquired points. The boundary may be a 2D boundary, such as a border, or a 3D boundary, such as a plane or a surface mesh. For example, the controller may assume the surface of the bone is locally flat and determine a border. The controller may determine a best fitting plane, project the point cloud on the best fitting plane, and compute the envelope of the projected points (e.g., with a standard convex hull determination algorithm or a concave hull extraction algorithm) to determine a boundary (e.g., a border or a plane). The controller may reconstruct a surface mesh from the point cloud (e.g., with a standard Delaunay triangulation algorithm or a Poisson Surface Reconstruction algorithm). The controller may extract a triangular surface boundary from the surface mesh.

The controller may be configured to determine a landmark point, for example the controller may extract one of the acquired points and deem it the landmark point (e.g., by virtue of its relation to the likely anatomical position of the landmark (e.g., an anatomical place). The controller may be configured to compare a distance between the boundary and the landmark point to a predetermined threshold. If the distance between the boundary and the landmark point is within the predetermined threshold, the landmark point is stored as the landmark point (and may be said to be robust). If the distance between the boundary and the landmark point is not within the predetermined threshold, the controller may be further configured to generate an indication requiring confirmation of the landmark point or a suggestion to acquire additional data points.

In some embodiments, the boundary is a border defined by edges of the point cloud. A distance between the border and the landmark point may not be within the predetermined threshold if the distance is less than the threshold. For example, it is useful to check whether the most distal or most posterior point extracted from the point cloud is too close to the border. If the distance to the border is below a given threshold, the controller generates an indication such as a warning or a suggestion to acquire points over a larger anatomical region.

The controller may be further configured to receive positional information comprising an acquired subsequent data point, determine an updated point cloud based on the plurality of acquired points and the subsequent acquired point, and determine an updated border associated with the updated point cloud. If the distance between the updated border and the landmark point is within the predetermined threshold, the landmark point is stored (and may be said to be robust). The controller may reiterate this process (warn, receive new acquired points, update the point cloud, update the border, compare to the predetermined threshold, etc.) until a landmark point is stored as the landmark point. In other words, storing the extracted landmark point as the landmark point indicates sufficient coverage of the likely anatomical position of the landmark.

In some instances, the previously extracted landmark point may be replaced with a subsequent acquired point (e.g., a more distal or more posterior point (e.g., more aligned with the desired anatomical landmark)). However, the extracted landmark point may not necessarily be replaced as a result of the addition of subsequent acquired points. In fact, the process may confirm that the formerly extracted landmark point was indeed the most distal or most posterior point, thus confirming sufficient coverage of the likely anatomical position of the landmark. Although FIGS. 3A and 3B depict a border as the boundary, it is understood that the boundary could be a plane.

The controller may use the stored landmark for intra-operative planning, as part of a coordinate system, for guidance of a robotic arm, etc.

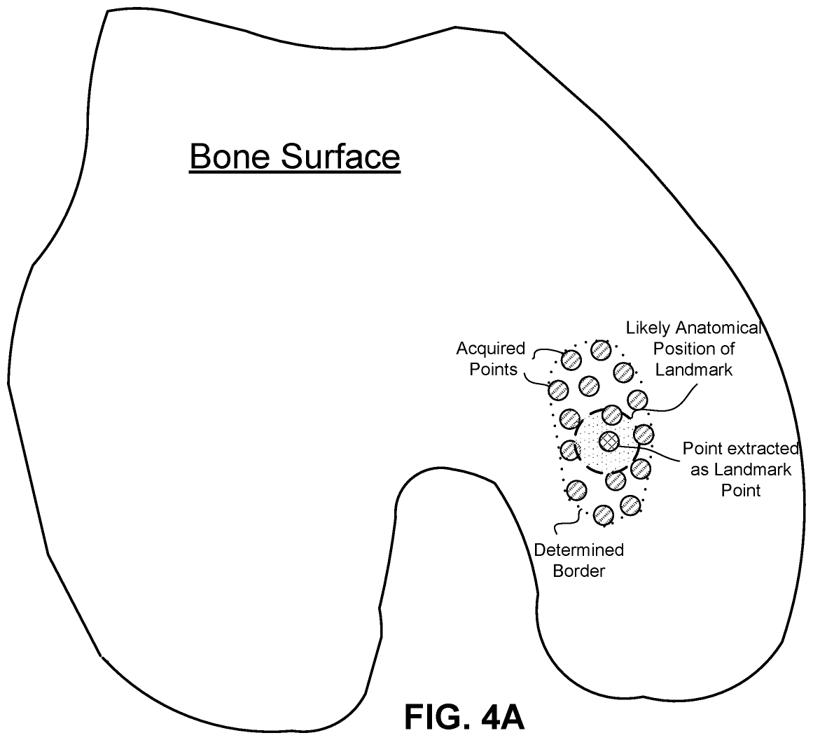

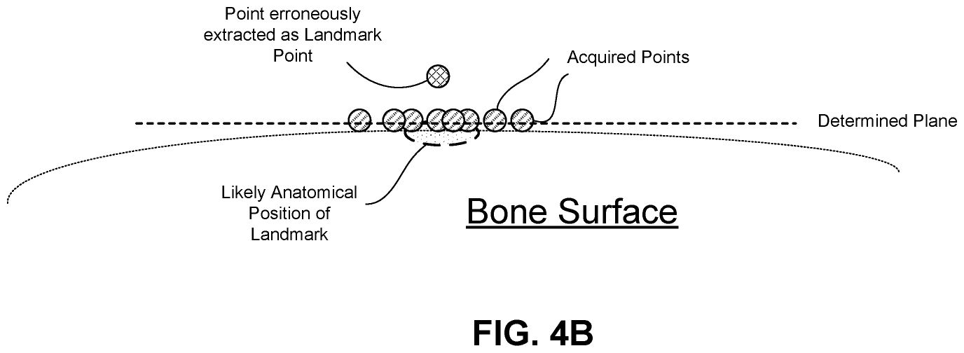

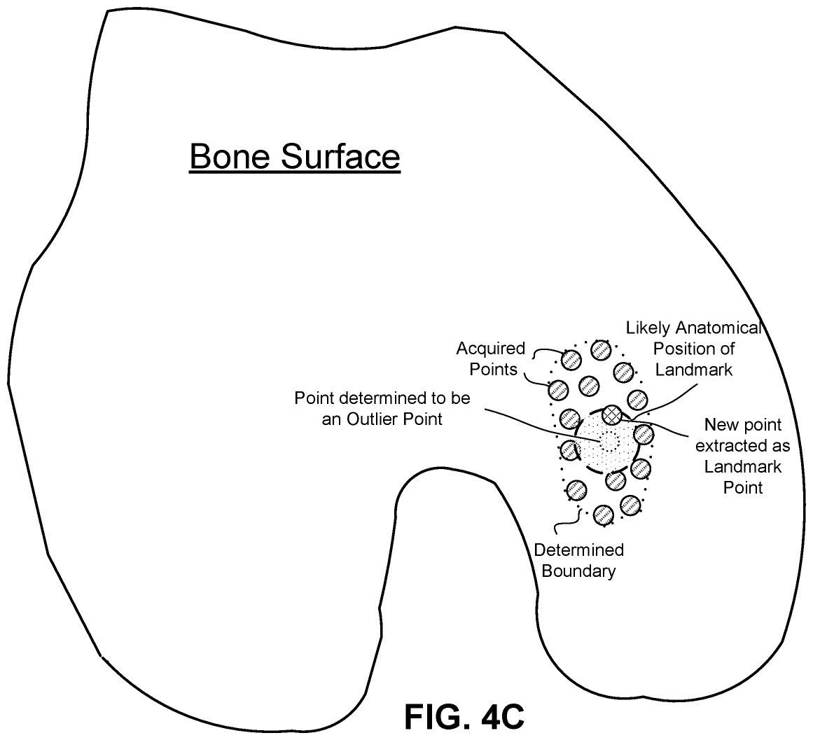

Turning to FIG. 4A, a landmark point is determined, for example, substantially as described above with respect to FIGS. 3A and 3B. During a contact-based process of acquiring points, a surgeon should constantly keep the pointer tool tip in contact with the bone surface. However, for various reasons, sometimes this contact is not maintained during the whole acquisition, or the surgeon might not realize that the pointer tip is actually in contact with another structure, such as soft tissues. In such a case, this might lead to a spurious point, hereinafter called an outlier. As may be appreciated, if the outlier is extracted as the landmark point, all uses of the landmark point thereafter will inherit this error. As may be appreciated, if a surgeon uses a contact-less acquisition method (e.g., laser or white light scanners for instance), a spurious point may correspond to noise, but would still be an outlier as described herein.

Turning to FIG. 4B, the landmark point is not an acquired point from the bone surface. Stated differently, an outlier point was erroneously extracted as the landmark point. Such a point is not, for example in the context of a UKA procedure, the most distal or most posterior point of the femur condyle.

To avoid this, in some embodiments, the boundary is determined as a plane. For example, a condyle surface is expected to have a smooth and continuous surface. Single points or portions of the point cloud that are isolated from the plane may be outliers. Single points or portions of the point cloud that represent local surface discontinuities may be outliers. The controller may employ an algorithm to determine the plane (e.g., a plane determination algorithm, for example a Least Squares approach). The plane will define a smooth surface that comprises acquired points that are implicitly connected to each other. The controller may be configured to determine whether a minimum point density is satisfied.

Alternatively, the controller may be configured to determine a boundary that is a surface mesh defined by the point cloud. The controller may employ a Poisson Surface Reconstruction algorithm to determine, for example, a triangular mesh. The controller may be configured to compare a surface location of the landmark point with surface locations of the other acquired points in the plurality of acquired points. The controller may be configured to determine a watertight surface (e.g., mesh) that interpolates all the points as best as possible while remaining smooth.

Regardless of boundary, the controller may be configured to ignore acquired points if the distance from plane (or from the interpolated surface mesh) to the acquired point is greater than the predetermined threshold. The controller may be configured to ignore isolated clusters of acquired points. For example, clusters may indicate structure that potentially belongs to other anatomical features. The controller may be configured to determine respective distances between the landmark point and each of the acquired points in the plurality of acquired points, wherein if the distance between the landmark point and each of the acquired points is not within a second predetermined threshold, the controller is further configured to generate an indication requiring confirmation of the landmark point, a suggestion to acquire additional points, or simply remove the landmark point.

The controller may be configured to determine a boundary that is a plane (or an interpolated surface mesh) defined by the point cloud. The controller may be configured to compare the distance between the plane (or mesh) and the landmark point to a predetermined threshold, and if the distance is greater than the threshold, the distance is not within the predetermined threshold.

Turning to FIG. 4C, if the distance between the plane (or mesh) and the landmark point is not within the predetermined threshold, the controller may be further configured to recalculate the plane defined by the point cloud by removing the landmark point. If the distance between the plane (or mesh) and the landmark point is not within the predetermined threshold, the controller may be further configured to extract a second point from the plurality of acquired points as indicating the landmark on the anatomical feature.

If the distance between the boundary and the landmark point is not within the predetermined threshold, the controller may be further configured to generate an indication requiring confirmation of the landmark point or a suggestion to acquire additional points. The controller may be further configured to receive positional information comprising an acquired subsequent data point, determine an updated point cloud based on the plurality of acquired points and the subsequent acquired point, and determine an updated plane associated with the updated point cloud. If the distance between the updated plane and the landmark point is within the predetermined threshold, the landmark point is stored (and may be said to be robust). The controller may reiterate this process (warn, receive new acquired points, update the point cloud, update the plane, compare to the predetermined threshold, etc.) until a landmark point is stored as the landmark point. In other words, storing the extracted landmark point as the landmark point indicates sufficient coverage of the likely anatomical position of the landmark.

In some instances, the previously extracted landmark point may be replaced with a subsequent acquired point (e.g., a more distal or more posterior point (e.g., more aligned with the desired anatomical landmark)). However, the extracted landmark point may not necessarily be replaced as a result of the addition of subsequent acquired points.

The controller may be configured to compare a collection order associated with acquisition of the plurality of acquired points. The controller may consider points in the order they are acquired and detect a trend. For example, the distances may increase (over the acquisition) as the pointer tool lifts off the surface. Analyzing this trend in the evolution of distance values may help to determine that a lift-off occurred, and even which points likely belong to the surface, and which ones are likely spurious. A large threshold may be used to detect a lift off with a high level of confidence. Analysis of how distances to the plane (or mesh) are increasing during collection may help to determine precisely when the pointer was not in contact with the surface anymore (e.g., with a finer level of detection).

In any of the foregoing embodiments, the controller may be configured to display (such as on the display 150 (FIG. 1B)) feedback, such as specific messages for efficiency. Additionally, the controller may be configured to display graphics, such as user tutorial, where potential lift off has happened, or where the bone acquisition coverage may be insufficient. The controller may be configured to display a popup or a similar mechanism to ask confirmation from the surgeon. The controller may be configured to allow the surgeon to proceed regardless of the robustness of the landmark.

The systems and methods described herein may be utilized in various applications involving robotic, robot-assisted, and non-robotic systems for surgical approaches and/or operations.

In an embodiment, a surgical system according to the present disclosure comprises a pointer, a navigation array attached to the pointer, a tracking system to detect and track elements of the navigation array, and a controller having at least one processor configured to: receive data from the pointer, receive pointer positional data from the tracking system to determine a three-dimensional position and orientation of the pointer, correlate the positional data to a position on a patient's bone surface, store the patient positional data as an acquired point, determine a point cloud based on the plurality of acquired points, determine a boundary associated with the plurality of acquired points, extract a first acquired point from the plurality of acquired points as indicating a landmark on an anatomical feature of the bone surface, compare a distance between the boundary and the landmark point to a predetermined threshold, and, if the distance between the boundary and the landmark point is within the predetermined threshold, store the extracted landmark point. The system may further comprise a second navigation array affixed to the patient's bone (bone array) or a surgical surface. The controller may be further configured to convey a warning if the landmark point is not within the predetermined threshold. The controller may be further configured to convey a suggestion to acquire more points if the landmark point is not within the predetermined threshold.

In another embodiment, a surgical system according to the present disclosure comprises a display, and a processor configured to: receive positional information comprising a plurality of acquired data points associated with a surface of an anatomical feature of a patient, determine a point cloud based on the plurality of data points, extract a point from the plurality of data points as indicating a landmark on the anatomical feature, determine a boundary associated with the plurality of data points, compare a distance between the boundary and the landmark point to a predetermined threshold, wherein if the distance between the boundary and the landmark point is not within the predetermined threshold, the processor is further configured to: generate an indication requiring confirmation of the landmark point or a suggestion to acquire additional data points.

The boundary may be a border defined by edges of the point cloud. The distance between the border and the landmark point is not within the predetermined threshold if the distance is less than the threshold. The processor may be further configured to receive positional information comprising an acquired subsequent data point, determine an updated point cloud based on the plurality of data points and the subsequent data point, and determine an updated border associated with the updated point cloud.

The boundary may be a plane defined by the point cloud. The distance between the plane and the landmark point is not within the predetermined threshold if the distance is greater than the threshold. If the distance between the plane and the landmark point is not within the predetermined threshold, the processor may be further configured to extract a second point from the plurality of data points as indicating the landmark on the anatomical feature. If the distance between the plane and the landmark point is not within the predetermined threshold, the processor may be further configured to recalculate the plane defined by the point cloud by removing the landmark point. The processor may be further configured to receive positional information comprising an acquired subsequent data point, determine an updated point cloud based on the plurality of data points and the subsequent data point, and determine an updated plane associated with the updated point cloud.

The boundary may be a surface mesh defined by the point cloud.

The processor may be further configured to compare a surface location of the landmark point with surface locations of the other data points in the plurality of data points. The processor may be further configured to: determine respective distances between the landmark point and each of the data points in the plurality of data points, wherein if the distance between the landmark point and each of the data points is not within a second predetermined threshold, the processor is further configured to: generate an indication requiring confirmation of the landmark point or a suggestion to acquire additional data points, or remove the landmark point.

The processor may be further configured to compare a collection order associated with acquisition of the plurality of data points.

In another embodiment, a method according to the present disclosure comprises: receiving positional information comprising a plurality of acquired data points associated with a surface of an anatomical feature of a patient, determining a point cloud based on the plurality of data points, extracting a point from the plurality of data points as indicating a landmark on the anatomical feature, determining a boundary associated with the plurality of data points, comparing a distance between the boundary and the landmark point to a predetermined threshold, and if the distance between the boundary and the landmark point is within the predetermined threshold, storing the landmark point, and if the distance between the boundary and the landmark point is not within the predetermined threshold, generating an indication requiring confirmation of the landmark point or a suggestion to acquire additional data points.

The boundary may be a border defined by edges of the point cloud and the distance between the border and the landmark point is not within the predetermined threshold if the distance is less than the threshold.

The boundary may be a plane defined by the point cloud and the distance between the plane and the landmark point is not within the predetermined threshold if the distance is greater than the threshold.

The method may further comprise: receiving positional information comprising an acquired subsequent data point, determining an updated point cloud based on the plurality of data points and the subsequent data point, and determining an updated boundary associated with the updated point cloud.

If the distance between the plane and a first landmark point is not within the predetermined threshold, the method may further comprise performing at least one of: ignoring the first landmark point and extracting a second point from the plurality of data points as indicating the landmark on the anatomical feature, or removing the first landmark point and recalculating the plane defined by the point cloud.

The method may further comprise comparing a surface location of the landmark point with surface locations of the other data points in the plurality of data points.

The method may further comprise determining respective distances between the landmark point and each of the data points in the plurality of data points, and if the distance between the landmark point and each of the data points is not within a second predetermined threshold, performing at least one of: generating an indication requiring confirmation of the landmark point, generating an indication suggesting a user acquire additional data points, or removing the landmark point.

1. A surgical system, the surgical system comprising:
a display; and
a processor configured to:
receive positional information comprising a plurality of acquired data points associated with a surface of an anatomical feature of a patient;
determine a point cloud based on the plurality of data points;
extract a point from the plurality of data points as indicating a landmark on the anatomical feature;
determine a boundary associated with the plurality of data points;
compare a distance between the boundary and the landmark point to a predetermined threshold, wherein if the distance between the boundary and the landmark point is not within the predetermined threshold, the processor is further configured to:
generate an indication requiring confirmation of the landmark point or a suggestion to acquire additional data points.

2. The surgical system of claim 1, wherein the boundary is a border defined by edges of the point cloud.

3. The surgical system of claim 2, wherein, the distance between the border and the landmark point is not within the predetermined threshold if the distance is less than the threshold.

4. The surgical system of claim 3, wherein the processor is further configured to:
receive positional information comprising an acquired subsequent data point;
determine an updated point cloud based on the plurality of data points and the subsequent data point; and
determine an updated border associated with the updated point cloud.

5. The surgical system of claim 1, wherein boundary is a plane defined by the point cloud.

6. The surgical system of claim 5, wherein, the distance between the plane and the landmark point is not within the predetermined threshold if the distance is greater than the threshold.

7. The surgical system of claim 6, wherein, if the distance between the plane and the landmark point is not within the predetermined threshold, the processor is further configured to extract a second point from the plurality of data points as indicating the landmark on the anatomical feature.

8. The surgical system of claim 6, wherein, if the distance between the plane and the landmark point is not within the predetermined threshold, the processor is further configured to recalculate the plane defined by the point cloud by removing the landmark point.

9. The surgical system of claim 6, wherein the processor is further configured to:
receive positional information comprising an acquired subsequent data point;
determine an updated point cloud based on the plurality of data points and the subsequent data point; and
determine an updated plane associated with the updated point cloud.

10. The surgical system of claim 1, wherein boundary is a surface mesh defined by the point cloud.

11. The surgical system of claim 1, wherein the processor is further configured to compare a surface location of the landmark point with surface locations of the other data points in the plurality of data points.

12. The surgical system of claim 11, wherein the processor is further configured to:
determine respective distances between the landmark point and each of the data points in the plurality of data points;
wherein if the distance between the landmark point and each of the data points is not within a second predetermined threshold, the processor is further configured to:
generate an indication requiring confirmation of the landmark point or a suggestion to acquire additional data points; or
remove the landmark point.

13. The surgical system of claim 1, wherein the processor is further configured to compare a collection order associated with acquisition of the plurality of data points.

14. A method, comprising:
receiving positional information comprising a plurality of acquired data points associated with a surface of an anatomical feature of a patient;
determining a point cloud based on the plurality of data points;
extracting a point from the plurality of data points as indicating a landmark on the anatomical feature;
determining a boundary associated with the plurality of data points;
comparing a distance between the boundary and the landmark point to a predetermined threshold; and
if the distance between the boundary and the landmark point is within the predetermined threshold, storing the landmark point; and
if the distance between the boundary and the landmark point is not within the predetermined threshold, generating an indication requiring confirmation of the landmark point or a suggestion to acquire additional data points.

15. The method of claim 14, wherein the boundary is a border defined by edges of the point cloud and the distance between the border and the landmark point is not within the predetermined threshold if the distance is less than the threshold.

16. The method of claim 14, wherein boundary is a plane defined by the point cloud and the distance between the plane and the landmark point is not within the predetermined threshold if the distance is greater than the threshold.

17. The method of claim 14, further comprising:
receiving positional information comprising an acquired subsequent data point;
determining an updated point cloud based on the plurality of data points and the subsequent data point; and
determining an updated boundary associated with the updated point cloud.

18. The method of claim 14, wherein, if the distance between the plane and a first landmark point is not within the predetermined threshold, performing at least one of:
ignoring the first landmark point and extracting a second point from the plurality of data points as indicating the landmark on the anatomical feature; or
removing the first landmark point and recalculating the plane defined by the point cloud.

19. The method of claim 14, further comprising comparing a surface location of the landmark point with surface locations of the other data points in the plurality of data points.

20. The method of claim 14, further comprising determining respective distances between the landmark point and each of the data points in the plurality of data points; and
if the distance between the landmark point and each of the data points is not within a second predetermined threshold, performing at least one of:
generating an indication requiring confirmation of the landmark point;
generating an indication suggesting a user acquire additional data points; or
removing the landmark point.
